(19) United States
(12) Patent Application Publication
(10) Pub. No.: US 2019/0321061 A1
(43) Pub. Date: Oct. 24, 2019

(54) Ultrasound Vessel Preparation

(71) Applicant: Justin Panian, San Francisco, CA (US)

(72) Inventor: Justin Panian, San Francisco, CA (US)

(21) Appl. No.: 16/034,685

(22) Filed: Jul. 13, 2018

Related U.S. Application Data
(60) Provisional application No. 62/661,222, filed on Apr. 23, 2018.

(51) Int. Cl. A61B 17/22

(52) U.S. Cl. CPC A61B 17/22012; A61B 2217/007; A61B 2017/22038; A61B 2017/22015

(57) ABSTRACT

Methods and devices are provided for vessel preparation to facilitate adjunctive therapies including the delivery of therapeutic drugs. An ultrasound catheter is provided, which has an ultrasound transmission member or wire with an affixed tip on the distal end, and an attached sonic connector on the proximal end extending through the catheter shaft which has a distal end coaxially positioned inside the tip and affixed to the tip. The ultrasound device propagates ultrasound energy while undergoing irrigation.

### FIELD OF THE INVENTION

The present invention is related to endovascular methods and devices for the treatment of stenosis and restenosis. More specifically, the present invention provides methods and devices for changing vessel compliance and for preparing the vessel for additional therapies, including the delivery of therapeutic drugs.

### BACKGROUND OF THE INVENTION

Atherosclerosis represent a major health problem throughout the world. A common treatment involves balloon angioplasty and stenting, a procedure in which a balloon catheter is advanced through the stenotic or occluded site and expanded there to widen the artery, and then a stent is placed at the treatment site for maintaining patency of the newly opened artery. Recently, Drug Eluding Stents (DES) and Drug Eluting Balloons (DEB) have been used to improve the clinical outcome to treat endovascular stenosis and re-stenosis. However, poor clinical outcomes are still observed in calcific arteries where delivering therapeutic drugs to the vessel wall is challenging because calcium build-up in media-intimal vessel wall is a major barrier that significantly prevents therapeutic drugs form reaching the vessel wall. While luminal calcification may be removed relatively easily by atherectomy devices, media-intima calcium is out of the reach of atherectomy devices, and impossible to remove because of high risk of vessel perforation.

Several efforts to address media-intima calcification, including the use of electromagnetic shock waves (ShockWave Medical, Fremont Calif.), ultrasound vibrational energy (CardioProlific Inc., Hayward, Calif.), and Excimer Laser (Philips, San Diego, Calif.) were tried but no commercial product has been introduced yet. Therefore, there is a need for effective methods for vessel preparation that would modify media-intima calcification for a follow up therapy including therapeutic drug delivery to the vessel wall.

### BRIEF SUMMARY OF THE INVENTION

Ultrasound is routinely used for diagnostic imaging applications, and lately has been adopted more often in various drug delivery and other therapeutic applications. Ultrasound has been shown to facilitate the delivery of drugs across the skin, promote gene therapy to targeted tissues, deliver chemotherapeutic drugs into tumors, and deliver thrombolytic drugs into blood clots, as well as the healing of wounds and bone fractures.

An ultrasound catheter of the present invention comprises an ultrasound transmission member or wire with an affixed tip on the distal end and an attached sonic connector on the proximal end extending through the catheter shaft which has a distal end coaxially positioned inside the tip and affixed to the tip. The ultrasound device propagates ultrasound energy while undergoing irrigation.

In one embodiment, the ultrasound catheter has a guidewire lumen extending inside the tip and affixed to the tip.

In another embodiment at least one irrigation outlet port is formed on the side of the tip and at least one irrigation outlet is formed longitudinally through the tip.

In yet another embodiment, ultrasound energy is propagated from the ultrasound catheter at a frequency between 1 KHz-20 MHz and energy intensity at the tip of more than 0.020 Watts.

In another embodiment, the tip of the ultrasound catheter has at least one side hole to affix the catheter shaft and at least one hole to affix the guidewire lumen.

In another embodiment, ultrasound energy propagates beyond the tip through saline, contrast, blood or a mixture of all, in the form of longitudinal waves.

In yet another embodiment, the ultrasound catheter is used for vessel preparation which may include treatment of stenosis, inhibit restenosis, plaque removal, thrombus removal, crossing totally occluded arteries or veins, treatment of vulnerable plaque, and may aide other therapies including therapeutic drug delivery.

In another embodiment, methods include advancing a distal end of an ultrasound catheter to an area of stenosis or restenosis in an artery or vein, and delivering ultrasound energy to change compliance of a surrounding plaque and calcium, thereby preparing a treatment side to deliver anti-stenotic therapeutic drugs.

In other embodiments of the present invention, devices and methods are provided utilizing ultrasound energy propagated in the form of longitudinal waves to change compliance of plaque or other vessel obstruction, and to increase the penetration of therapeutic drugs to the vessel wall. Ultrasound energy can moderate and fracture calcified plaque while causing minimal injury to a healthy tissue, Application of ultrasound longitudinal waves may induce micro-cracks in the plaque and create micro-channels to further facilitate therapeutic drug penetration and increase permeability of the vessel.

In another embodiment, an ultrasound transducer includes a housing enclosure containing a dual sleeve assembly disposed around the transducer horn comprising an outer sleeve and an inner sleeve, a catheter knob which is inserted in to the distal end of the transducer housing and positioned between the inner and outer sleeves, Positioning the knob inside the transducer housing between inner and outer sleeves is configured to mitigate unwanted bending stress on the connection of the catheter and the transducer.

In another embodiment, devices of the present invention are suitable for crossing chronic total occlusion and if needed, perform atherectomy to facilitate the introduction of a balloon catheter to the treatment area.

Methods of the present invention include providing an ultrasound catheter to the treatment area, delivering ultrasound longitudinal waves to perform vessel preparation, crossing chronic total occlusion, and performing atherectomy.

### BRIEF DESCRIPTION OF DRAWINGS

FIG. 1 is a schematic overall view of an ultrasound catheter according to an embodiment of the present invention,

FIG. 2 is an enlarged cross-sectional view of the distal tip of the catheter shown in FIG. 1,

FIG. 3 show the catheter of FIG. 1 delivering ultrasound energy to the treatment area.

FIG. 4 shows the vessel of FIG. 3 after exposure to ultrasound energy.

FIG. 5 shows a transducer enclosure with dual sleeves.

FIG. 6 shows a knob assembly configured distally as a sleeve.

FIG. 7 shows a knob positioned inside the transducer enclosure of FIG. 5.

FIGS. 8A and 8B show a sonic connector having a flat thread.

### DETAILED DESCRIPTION OF THE DRAWINGS

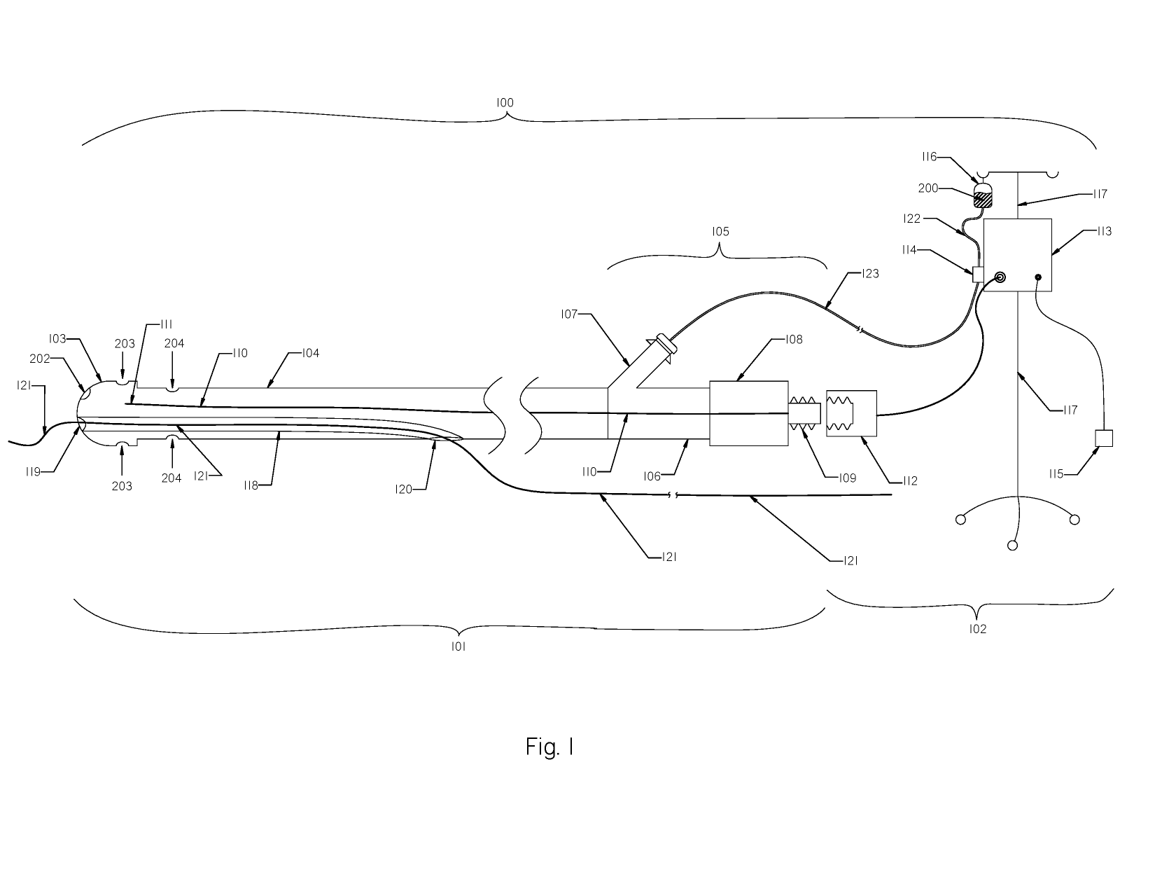

FIG. 1 shows an overall view of an ultrasound system 100 comprising an ultrasound catheter 101 and an ultrasound generating device 102. The ultrasound catheter 101 comprises a tip 103, a catheter shaft 104 and a proximal assembly 105. The proximal assembly 105 comprises a Y-connector 106 having an irrigation inlet port 107 and a knob 108. The proximal assembly 105 also includes a sonic connector 109 attached to the proximal end of an ultrasound transmission member 110 and connectable to a transducer assembly 112 which is a part of the external ultrasound generating device 102. The ultrasound transmission member 110 is affixed inside the tip 103 at location 111.

The ultrasound generating device 102 comprises a transducer assembly 112, an ultrasound generator 113, an irrigation pump 114, a footswitch 115 and an irrigation bag 116 that contains irrigant 200 (e.g., NaCl—Sodium Chloride Solution 0.9%). The bag 116 with the irrigant 200 is placed on an IV pole 117.

A guidewire lumen 118 having a distal end 119 is extended through the tip 103 of the catheter shaft 104. A proximal end of the guidewire lumen 118 extends through the catheter shaft 104 at an exit port 120. As an example, a guidewire 121 can be extended through the distal end 119 of the guidewire lumen 118 and exits through the exit port 120.

The irrigation pump 114 is connected to the irrigant bag 116 via a tube 122 and to the irrigation inlet port 107 of the Y-connector 106 via a tube 123. The irrigation pump 114 forces delivery of irrigant 200 from the bag 116 to the irrigation inlet port 107, through the catheter shaft 104 to the distal tip 103. At least one irrigant outlet 202 is longitudinally extended through the tip 103. Alternatively, or in addition, irrigation exits ports 203 may be positioned radially on the tip 103 and yet another irrigant outlet port 204 may be located on the catheter shaft 104. Irrigant outlet ports locations and numbers may vary, and outlet ports may be placed as needed at any location on the tip 103 and/or along the catheter shaft 104.

Ultrasound energy generated by the transducer assembly 112 propagates via the sonic connector 109 and through the ultrasound transmission member 110 to the tip 103. As a result, the tip 103 undergoes vibrational motion that further propagates ultrasound waves outside the tip 103.

The catheter shaft 104 may be formed of any suitable material, including but not limited to flexible polymeric materials. The flexible catheter shaft 104 is generally in the form of an elongate tube having one or more lumens extending longitudinally therethrough. The catheter lumen 118 may be made of the same or similar polymeric materials.

The distal tip 103 is a rigid member firmly affixed to the ultrasound transmission member 110 at attachment location 111 and affixed to the catheter shaft 104 by any suitable method, including but not limited to, gluing or fusing. The distal tip 103 has a generally rounded configuration and may be formed of any suitable rigid metal or plastic material, preferably radio-dense material to be better visible by radiographic means.

The tip 103 is attached to the ultrasound transmission member 110 at the attachment area 111 by welding, adhesive, soldering, crimping, or by any other suitable means. A firm affixation of the ultrasound transmission member 110 to the distal tip 103 and the sonic connector 109 is required for continuous and un-interrupted vibrational energy transmission from the transducer assembly 112 to the tip 103.

The ultrasound transmission member 110 may be formed of any material capable of effectively transmitting the ultrasonic energy including but not limited to metal, fiber optics, polymers, and/or composites. The ultrasound transmission member 110 may be formed of one or more Nickel/Titanium alloys also known as shape memory or super elastic alloys. Super-elastic metal alloys are known in the art and will not be further specified. The ultrasound transmission member may include one or more wires, tubes or combination of both.

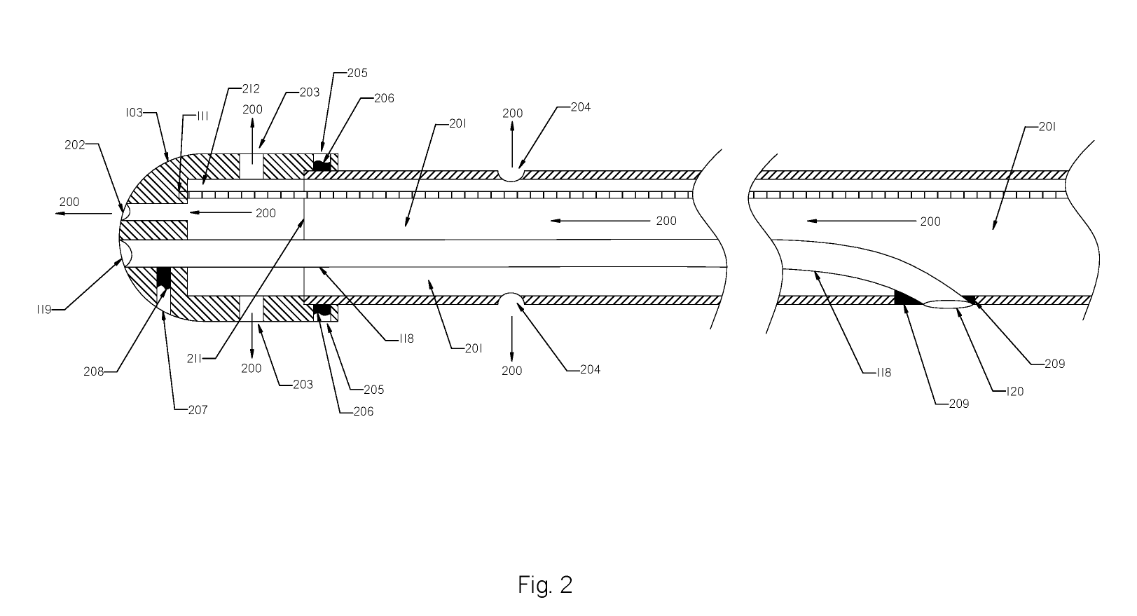

FIG. 2 shows the tip 103 of FIG. 1. Irrigant 200 is delivered from the irrigation bag 116 using the pump 114 via the irrigation tube 123 and the irrigation inlet 107 of the Y-connector 106 to the inner lumen 201 of the catheter shaft 104 and to the distal tip 103. Irrigant 200 may exit the ultrasound catheter 101 via the exit port 202 and/or the exit port 203 located on the tip 103 and/or the exit port 204 located on the catheter shaft 104. Irrigant 200 is often referred to as coolant fluid (e.g., 0.9% NaCl solution) that serves to prevent overheating of the ultrasound member 110 during ultrasound energy delivery.

After exit from the catheter shaft 104 through outlet exits 202, 203 and 204, the irrigant 200 surrounds and baths the tip 103 and provides a vehicle for ultrasound waves to propagate outside the tip 103.

The tip 103 may include one or more perpendicular holes 205 that provide access from the outside of the tip 103 to the shaft 104 located inside the tip 103. The hole(s) 205 is constructed for the application of glue 206 and bonding the tip 103 with the catheter shaft 104. Application of glue 206 bonds the tip 103 to the catheter shaft 104 and affixes the tip 103 to the catheter shaft 104. Alternatively, or in addition, another hole(s) 207 may be located on the distal part of the tip 103 which provides access from outside of the tip 103 to the guidewire lumen 118 located inside the tip 103. The hole 207 is constructed to glue 208 to he applied, and to bond the tip 103 with the guidewire lumen 118 located inside the tip 103. Application of glue 208 affixes the tip 103 to the guidewire lumen 118. Alternatively, the guidewire lumen 118 may be glued to the shaft 104 at any location within an interface between two these components, for example proximal to the tip 103 at location 209. The distal end 119 of the guidewire lumen 118 as shown in FIG. 2 is flush with the outer surface 210 of the tip 103. If desired, the distal end 119 of the guidewire lumen may be located inside the tip 103 (not shown) or extended distal to the outer surface 210 of the tip 103 (not shown).

The ultrasound member 110 is coaxially deposed inside the inner lumen 201 of the catheter shaft 104 and affixed off-center to the tip 103, inside the tip 103 at the attachment area 111. Attachment methods may include, but are not limited to, welding, soldering, fusing, gluing, thigh fitting, or combinations thereof. The distal end 211 of the catheter shaft 104 is coaxially disposed inside the bore 212 of the tip 103.

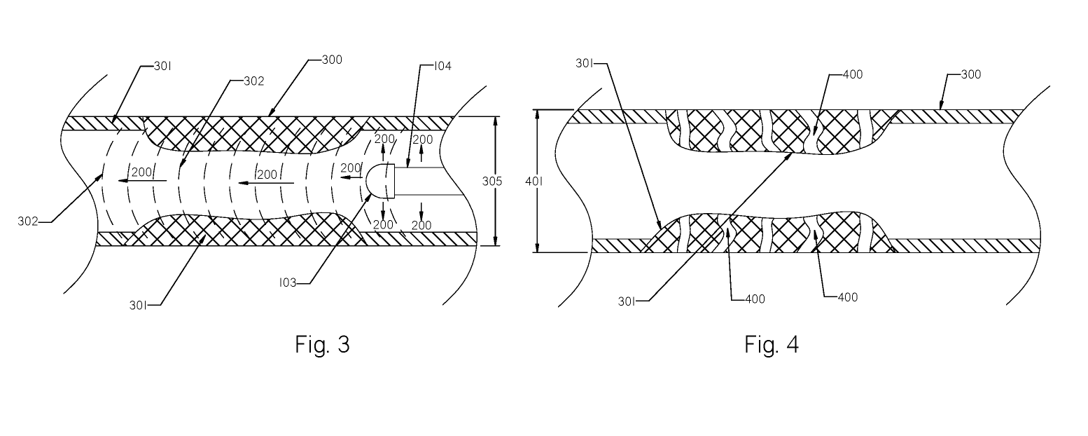

FIG. 3 shows the tip 103 of FIG. 1 and FIG. 2 located inside the vessel 300. The treatment area 301 of the vessel may include plaque, calcification, scared tissue, clots and combination thereof. Ultrasound energy is delivered from the vibrating tip 103 via irrigant 200 which immerses the tip 103 and provides a vehicle for ultrasound waves to further propagate outside the tip 103 and distally inside the vessel 300.

The longitudinal waves 302 oscillate in the longitudinal direction and are originated from the tip 103. While the distal tip oscillates in the direction of wave propagation, ultrasound waves also propagate radially around the tip 103. Longitudinal waves 302 are capable of propagating from the tip 103 through irrigant, blood or mixture of both to the treatment area 301 inside the vessel 300 and beyond distally and outside the vessel 300,

The longitudinal sound waves are known in the art to permanently disrupt the integrity of the hard plaque while being friendly to cell membranes, and do not create permanent damage to the vessel wall or surrounding healthy tissue. In a typical application, ultrasound energy in contact or in proximity to the treatment area 301 inside the vessel wall 300 is used at a frequency of about 20-100 kHz and a power of more than 0.020 Watts. Such power levels of ultrasound energy are suitable to cause vessel vasodilatation and increase vessel permeability, create micro-cracks and micro-channels in the plaque. Ultrasound energy power levels less than 0.020 Watts will not have such impact.

The tip 103 of the ultrasound catheter 101 may be repositioned distally or proximally around the treatment area 301. The tip 103 may be also moved back and forth at the treatment area 301 as desired. Such repositioning of the tip 103 and delivery of ultrasound longitudinal waves would cover a longer segment to be treated. Exposure of ultrasound energy to the treatment area may last anywhere from 1 second to one hour, depending on the plaque composition at the treatment area,

FIG. 4 shows the treatment area 301 of the vessel 300 after ultrasound energy has been applied, and after the ultrasound catheter 101 has been removed. Longitudinal waves 302 propagated from the vibrating tip 103 can modify the structure or change compliance of the treated area 301, inducing cracks and creating micro-channels 400. Also, longitudinal waves propagated from the vibrating tip 103 are capable of causing a transient vasodilatation which is shown by comparing the vessel's 300 original diameter 305 (see FIG. 3) which is increased to a larger size 401 (see FIG. 4) after the delivery of ultrasound energy delivery, Such transient vasodilatation may last 30 minutes or more, and after that period of time, the vessel will return to its original size (pre-ultrasound energy delivery). Transient vasodilatation will temporarily expand extracellular space at the treatment area and may increase vessel permeability.

Both these methods of action (inducing cellular changes by vasodilatation which may increase vessel permeability, and modifying compliance of the treatment area by inducing micro-cracks and creating micro-channels) may be helpful to further facilitate drug therapies. In the spirit of the present invention one of these methods or both these methods combined are considered as “vessel preparation”.

Therapeutic drugs may be delivered to a treatment area 301 before, during or after ultrasound vessel preparation, and may include delivering liquid drug directly to the treatment through irrigation holes and/or in mixture with irrigation. The irrigation fluid may be delivered via one or more outlet ports on the ultrasound catheter that are separate from one or more therapeutic agent outlet ports. Therapeutic drugs may also be delivered in a variety of other methods, including but not limited to, use of DES, DEB, Bioabsorbable Stents and systemic drug delivery.

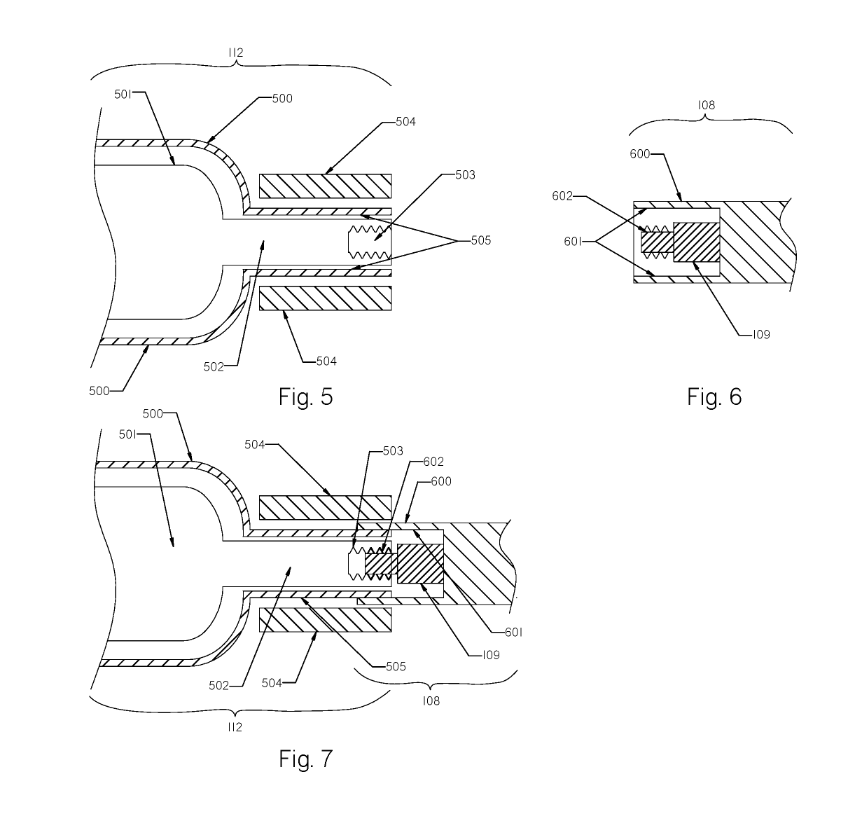

FIG. 5 shows the transducer assembly 112 of FIG. 1 comprising a transducer enclosure housing 500 containing a transducer 501 having a horn 502, The transducer assembly 112 is connected to a signal generator113 as shown in FIG. 1. The transducer horn 502 includes a female thread 503. Alternatively, the transducer horn 502 may include a male thread (not shown). The transducer housing 500 comprises an outer sleeve 504 and an inner sleeve 505 which are disposed around the transducer horn 502.

FIG. 6 shows the knob 108 of FIG. 1. The knob 108 comprises a proximal sleeve having an outer wall 600 and an inner wall 601. The sonic connector 109 is disposed inside the knob 108. The sonic connector 109 comprises a male thread 602 which serves as a connection between the sonic connector 109 and the transducer horn 502. Alternatively, the sonic connector 109 may include a female thread (not shown),

FIG. 7 shows the knob 108 of the FIG. 6 placed inside the transducer housing 500 of FIG. 5. The sonic connector109 is threadably connected inside the thread 503 of the transducer horn 502. The interior wall 601 of the knob 108 is placed over the inner sleeve 505 of the transducer housing 500 and under the outer sleeve 504 of the transducer housing 500. Such overlapping edifice between the knob 108 and inner sleeve 505 and outer sleeve 504 of the transducer housing 500 provides a secure interface between transducer housing 500 and the knob 118. Such overlapping interface between the knob 108 and the outer sleeve 504 and the inner sleeve 505 prevents and minimizes the impact of bending forces on the connection of the knob 118 and the transducer housing 500, thereby mitigating related and unwanted stress on the sonic connector 109 and the transducer horn 501 during the delivery of ultrasound energy. Consequently, ultrasound energy losses at the connection between the sonic connector 109 and the transducer horn 502 are reduced.

One significant challenge in the clinical field is related to connecting the sonic connector 109 to the transducer horn 502, because of the need for several 360-degree rotations of the sonic connector 109 to secure the sonic connector 109 to the transducer horn 502. During the process of connecting the sonic connector 109 to the transduce horn 502, the catheter 100 as shown in FIG. 1 must be placed in the sterilized field before connecting it to the transducer assembly 112. Considering the length of the catheter 100, often in the range of 50-300 cm, connection of the sonic connector 109 to the transducer horn 502 will cause multiple catheter rotations which may displace the ultrasound catheter 100 out of the sterilized field and often is time consuming.

Reducing the number of catheter rotations when connecting the sonic connector 109 to the transducer horn 502 would significantly improve clinical procedures. The male thread 602 may include a conventional screw configuration or may be modified and adopted to reduce the number of sonic connector 109 turns to connect the sonic connector 109 to the transducer horn 502.

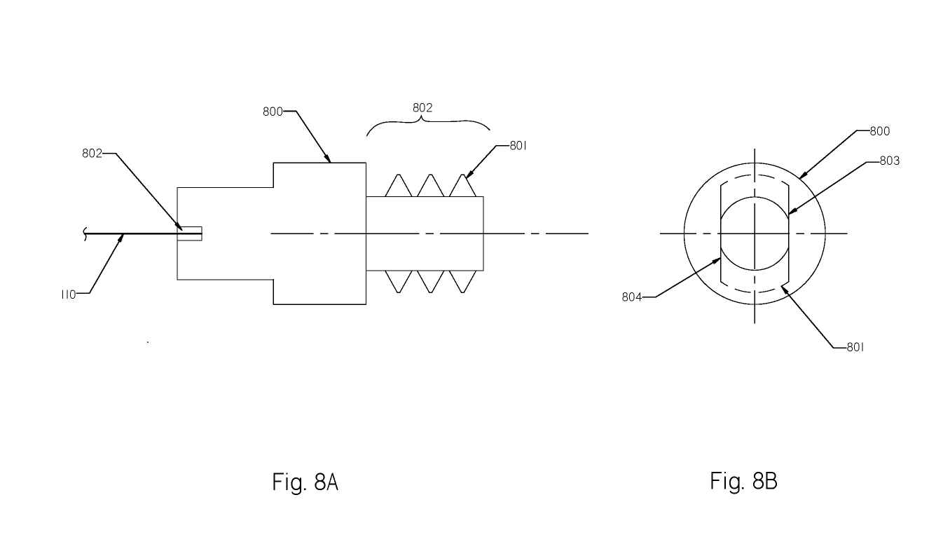

FIG. 8A and FIG. 8B show a sonic connector 800 that includes a flattened thread 801 on the proximal thread 802. Such flattened thread 801 comprises a flat side 803 and the flat side 803 that allows a quick engagement and disengagement of the sonic connector 800 into the inner thread 503 of the transducer horn 502. Such flattened thread configuration engages the sonic connector 109 into the transducer tread 503 more quickly because it has two thread engagement areas, thereby reducing the number of rotations required to securely screw the sonic connector 109 inside the tread 503.

Many other shapes and configurations of threads may be suitable to improve the connection between the sonic connector 109 and the transducer horn 502, and to reduce the number of turns to connect both while maintaining a secure connection, including but not limited to parallel flats, non-parallel flats, groves, slits, cuts. gashes, holes, and slashes among other.

The connection between the sonic connector 109 and transducer horn 502 that propagates ultrasound energy undergoes vibrational stress and related forces that are capable of shaking and unscrewing this connection during ultrasound energy delivery. Experimental work by the present inventor has revealed that if the torque connecting the sonic connector 109 to the transducer horn 502 before activation of ultrasound energy is less than 5 lb-in torque, such connection will be prone to fail and disconnect. In such cases, reconnecting the sonic connectors 109 with the transducer horn 502 would be required. While reconnecting of sonic connectors 109 to transducer horn 502 is a relatively simple step, it is often difficult to detect if these two components are disconnected or not, and such detection relays on harmonic frequencies created during such disconnection that may cause audible sound that are not always generated. Also, such re-connection may be required to be done several times during the clinical procedure and consequently, may result in poor device clinical performance.

Alternatively, the sonic connectors may comprise a luer locking fastener to further improve and facilitate connection to the transducer horn. Such fasteners are known in the art and often used for connecting syringes to other accessories such as tubes, Y-connectors, two-way valves, or three-way valves. Such luer locking fastener may comprise a radial flange, rim, collar, rib, lip, nozzle, protrusion or combinations thereof.

Methods of the present invention comprise providing an ultrasound catheter as shown in of FIG. 1 and FIG. 2, and delivering ultrasound longitudinal waves to the treatment area as shown in FIG. 3A and FIG. 3B to perform vessel preparation as shown in FIG. 4.

Other methods may include crossing chronic total occlusions and performing atherectomy utilizing the ultrasound catheter of the present invention. These methods are known in the art and will not be described in this specification.

Some theoretical considerations have been provided as to describe the mechanism by which these therapeutic methods are effective; these considerations have been provided only to conveying an understanding of the invention and have no relevance to or bearing on claims made to this invention.

1. An ultrasound catheter, comprising:
an ultrasound generating device;
a sonic connector that is connectable to the ultrasound generating device;
an elongated ultrasound transmission member having a distal end affixed to a tip and a proximal end affixed to the sonic connector;
a catheter shaft having a distal end and a proximal end, the catheter shaft extending around the transmission member, a guidewire lumen located inside the catheter shaft having a proximal end and a distal end which extends through the tip, wherein the distal end of the catheter shaft is positioned coaxially inside the tip and affixed to the tip, and wherein the ultrasound catheter propagates ultrasound energy to a treatment site while undergoing irrigation.

2. The catheter of claim 1, wherein the distal end of the guidewire lumen is terminated in one of the following locations: inside the tip, even with the tip, or extended beyond the tip.

3. The catheter of claim 1, wherein the ultrasound member is coaxially disposed inside the catheter shaft and affixed off-center to the tip.

4. The catheter of claim 1, wherein the ultrasound energy includes longitudinal waves.

5. The catheter of claim 1, wherein at least one irrigation outlet hole is formed in the following locations: on the side of the tip, longitudinally through the tip, in the catheter shaft, or a combination thereof.

6. The catheter of claim 1, wherein the ultrasound energy propagates at a frequency between 1 KHz-20 MHz, wherein energy intensity at the tip is more than 0.020 Watts.

7. The catheter of claim 1, wherein the distal end of the guidewire lumen is affixed to the tip, wherein the proximal end of the guidewire lumen exits the catheter shaft at an exit port along the catheter shaft, and wherein the guidewire lumen is further affixed to the catheter shaft.

8. The catheter of claim 1, wherein the tip has at least one side hole to affix the catheter shaft and wherein the tip has at least one hole to affix the guidewire lumen,

9. The catheter of claim 1, wherein treatment sites include endovascular locations, wherein the ultrasound energy is used for vessel preparation, and wherein vessel preparation comprises compliance change of the treatment area including inducing micro-cracks and creating micro-channels within the treatment area.

10. The catheter of claim 1, wherein the sonic connector comprises means for rapid connection to the transducer and rapid disconnection from the transducer, and wherein the means includes a flattened thread.

11. A system for connecting an ultrasound catheter and a transducer housing to propagate ultrasound energy comprising: an ultrasound catheter having a knob and a sonic connector, a transducer housing containing a transducer horn and comprising a dual sleeve assembly having an outer sleeve and an inner sleeve disposed around the transducer horn, wherein the sonic connector and transducer horn are threadably connected, and wherein the knob is inserted into the transducer housing and positioned between the inner sleeve and outer sleeve.

12. The system of claim 11, wherein the knob that is positioned inside the transducer housing between the inner sleeve and outer sleeve is configured to mitigate unwanted bending stress on the connection between the ultrasound catheter and the transducer.

13. The system of claim 11, wherein the torque of the threaded connection between the sonic connector and the transducer horn is more than 5 lb-in.
